(19) United States
(12) Patent Application Publication
(10) Pub. No.: US 2021/0153999 A1
(43) Pub. Date: May 27, 2021

(54) INTRA-AORTIC EMBOLIC PROTECTION FILTER DEVICE

(71) Applicant: FILTERLEX MEDICAL LTD., Yokneam (IL)

(72) Inventors: Sigalit ELI, Tel-Aviv (IL); Eyal TEICHMAN, Hod-Hasharon (IL)

(21) Appl. No.: 17/056,994

(22) PCT Filed: May 22, 2019
(86) PCT No.: PCT/IL2019/050579

§ 371 Date: Nov. 19, 2020

Related U.S. Application Data
(60) Provisional application No. 62/674,692, filed on May 22, 2018.

(51) Int. Cl. A61F 2/01

(52) U.S. Cl. CPC A61F 2/0105; A61F 2220/0008; A61F 2210/0014; A61F 2002/016; A61F 2250/0098; A61F 2230/0069; A61F 2/013; A61F 2230/0008

(57) ABSTRACT

An embolic protection filter device comprising: a deflector comprising a filter screen; an anchor comprising a cylinder-like frame having at least one filter pocket attached internally thereto, said filter pocket having an opening directed upstream; and a connecting section configured for connecting said deflector and said anchor.

### RELATED APPLICATIONS

This application claims the benefit of priority of U.S. Provisional Patent Application No. 62/674,692, filed on May 22, 2018.

This application is also related to U.S. Provisional Patent Application No. 62/215,075 and PCT Patent Application No. PCT/IL2016/050992, filed on Sep. 7, 2016 and published as WO/2017/042808.

The contents of the above applications are all incorporated by reference as if fully set forth herein in their entirety.

### FIELD AND BACKGROUND OF THE INVENTION

The present invention, in some embodiments thereof, relates to the field of intra-aortic embolic protection devices.

In transcatheter left heart procedures such as Aortic-Valve Implantation and/or Replacement (TAVI and TAVR, respectively), a catheter-based delivery system and compressed/crimped prosthetic valve may be inserted through one of the arteries and advanced to the aortic root. After careful positioning of the device in the native aortic valve, the new prosthetic valve may be deployed and may immediately function as a new aortic valve.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

There is provided, according to some embodiments, an embolic protection filter device comprising: a deflector comprising a filter screen; an anchor comprising a cylinder-like frame having at least one filter pocket attached internally thereto, said filter pocket having an opening directed upstream; and a connecting section configured for connecting said deflector and said anchor.

In some embodiments, the device comprises an integrally-formed frame structure, said frame structure being configured for collapsing, in a delivery state, into a reduced diameter configuration around a longitudinal axis thereof.

In some embodiments, the frame structure comprises a plurality of filaments arranged in a cell-like construction.

In some embodiments, the frame structure is made of one or more materials selected from the group consisting of: Nitinol, shape-memory metal alloy, metal spring alloy, stainless steel, titanium, titanium alloy, super-elastic material, and bio-compatible polymer.

In some embodiments, the deflector is configured for positioning within an aortic arch and for conforming to a superior wall of the aortic arch.

In some embodiments, the deflector is formed as an elongated trough which curves along a longitudinal axis thereof In some embodiments, a longitudinal center of said elongated trough is configured for providing a track for guiding surgical instruments traversing the aortic arch.

In some embodiments, the deflector further comprises a support frame, said support frame having an elongated oval-like shape.

In some embodiments, the support frame further comprises one or more notches at corresponding one or more internal corners thereof, said notches being configured for facilitating collapsing of said support frame along said longitudinal axis into said delivery state.

In some embodiments, the filter screen is attached to said support frame along a perimeter of said support frame.

In some embodiments, the support frame further comprises at least three radio-opaque markers at specified points along said perimeter, wherein an orientation of said positioning of said deflector within the aortic arch can be determined based, at least in part, on imaging said at least three radio-opaque markers.

In some embodiments, the anchor is configured for positioning within a descending aorta, and for radially expanding against a wall of the descending aorta so as to provide an anchor point for said device.

In some embodiments, the cylinder-like frame of said anchor comprises an obliquely-cut section at a downstream end thereof.

In some embodiments, the anchor comprises at least two filter pockets arranged longitudinally side-by-side internally thereof.

In some embodiments, the at least two filter pockets are configured for trapping emboli flowing downstream through said anchor, wherein said trapping causes an expansion of said at least two filter pockets.

In some embodiments, the expansion occurs, at least in part, within said obliquely-cut section.

In some embodiments, the openings of said at least two filter pockets are each attached along at least a portion of its perimeter to said cylinder-like frame, wherein said attaching causes said openings to maintain at least a partially-opened state.

In some embodiments, the blood flow through said openings, in said least partially-opened state, further causes said openings to achieve a fully-opened state.

In some embodiments, in said fully-opened state, the openings are configured for jointly covering substantially a cross-sectional area of said cylinder-like frame.

In some embodiments, the adjoining walls of said at least two filter pockets are at least partially attached to each other.

In some embodiments, the attaching of said adjoining walls is further configured to enable a surgical tool to pass through a gap between said adjoining walls, wherein said gap is dimensioned to effect a seal around a periphery of said surgical tool.

In some embodiments, downstream receptacle areas of said at least two filter pockets are located within said obliquely-cut area of said anchor.

In some embodiments, the connecting section comprises an elongated strip comprising at least two filaments arranged in said cell-like construction.

In some embodiments, the connecting section has a length of between 20 and 200 millimeters, and a width of between 5 mm to 60 mm.

In some embodiments, the connecting section has an arch-like cross-sectional profile.

There is also provide, in some embodiments, a method for protecting a patient against flow of emboli from an aorta to branching arteries, the method comprising providing a device comprising a deflector comprising a filter screen, an anchor comprising a cylinder-like frame having at least one filter pocket attached internally thereto, said filter pocket having an opening directed upstream, and a connecting section configured for connecting said deflector and said anchor; and inserting said device via a catheter into an aorta, such that said deflector screen is positioned at the aortic arch, and said anchor is positioned along the descending aorta.

In some embodiments, the positioning of said anchor along the descending aorta comprises said anchor self-expanding against a wall of said descending aorta, to provide an anchoring of said device.

In some embodiments, the positioning of said deflector at the aortic arch further comprises pushing said deflector, via said connecting section, against a superior wall of the aortic arch.

In some embodiments, the deflector further comprises a support frame comprising at least three radio-opaque markers at specified points along a perimeter thereof, the method further comprising the step of determining an orientation of said positioning of said deflector within the aortic arch based, at least in part, on imaging said at least three radio-opaque markers.

In some embodiments, the anchor comprises at least two filter pockets arranged longitudinally side-by-side internally thereof, wherein adjoining walls of said at least two filter pockets are at least partially attached to each other, so as to enable a surgical instrument to pass through a gap between said adjoining walls, the method further comprising performing a procedure on a heart of the patient by passing said surgical instrument through said gap.

In some embodiments, the deflector is formed as an elongated trough which curves along a longitudinal axis thereof, the method further comprising using a longitudinal center of said elongated trough as a track for guiding said surgical instruments while traversing the aortic arch.

In some embodiments, the step of collecting said device into said catheter, wherein said collecting comprises collapsing said device into a delivery state wherein said device has a reduced diameter configuration around a longitudinal axis thereof.

In addition to the exemplary aspects and embodiments described above, further aspects and embodiments will become apparent by reference to the figures and by study of the following detailed description.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified illustration of the intra-aortic emboli protection filter device deployed in an aorta, according to an exemplary embodiment of the invention;

FIGS. 2A-2D show an intra-aortic emboli protection filter device, according to an example embodiment of the invention;

FIG. 3A shows an anchor of an intra-aortic emboli protection filter device, according to an example embodiment of the invention;

FIGS. 3B-3C illustrate various views of filter pockets within an anchor of an intra-aortic emboli protection filter device, according to an example embodiment of the invention;

FIGS. 4A-4D illustrate various views of a deflector of an intra-aortic emboli protection filter device, according to an example embodiment of the invention;

FIG. 5 shows an exemplary connecting section of an intra-aortic emboli protection filter device, according to an example embodiment of the invention;

FIGS. 6A, 6B and 6C, are cross-section view simplified illustrations of a molding configuration of the filter mesh to the intra-aortic emboli protection filter device frame in accordance with some embodiments of the current invention;

FIGS. 7A and 7B, are perspective view and cross-section view simplified illustrations of an intra-aortic emboli protection filter device (7A) and a cross-section of an aorta (7B) in accordance with some embodiments of the current invention; and

FIGS. 8A, 8B and 8C, are cross-sections of an intra-aortic emboli protection filter device frame wire in accordance with some embodiments of the current invention.

### DETAILED DESCRIPTION

Disclosed herein is an intra-aortic emboli protection filter device and an associated method. In some embodiments, the present device provides for deflecting and/or capturing and/or removing of emboli particles dislodged into the blood stream during various cardiac left-heart interventional procedures.

Some examples of cardiac procedures which may potentially benefit from using an intra-aortic emboli protection device and methods as described herein include trans-catheter procedures, such as trans-catheter aortic valve implantation or replacement (TAVI/TAVR), atrial fibrillation ablation, left atrial appendage closure, and mitral valve repair and replacement. During TAVI/TAVR valve delivery, manipulation and deployment, for example, calcium particles may be dislodged from the stenotic native aortic valve and the surrounding vasculature to the vascular system. Together with aortic valve leaflets, collagenous and isolated thrombus, these particles might migrate to the brain and to other vital organs and cause ischemia-related damage to these organs.

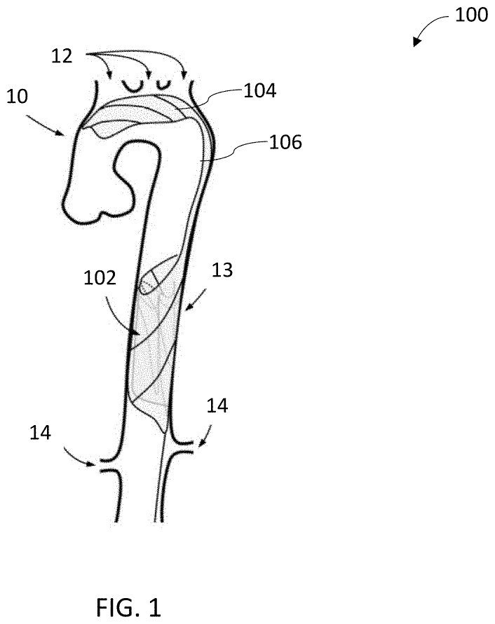

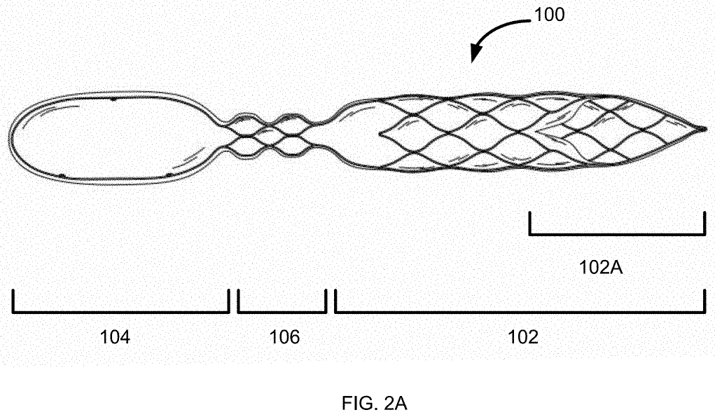

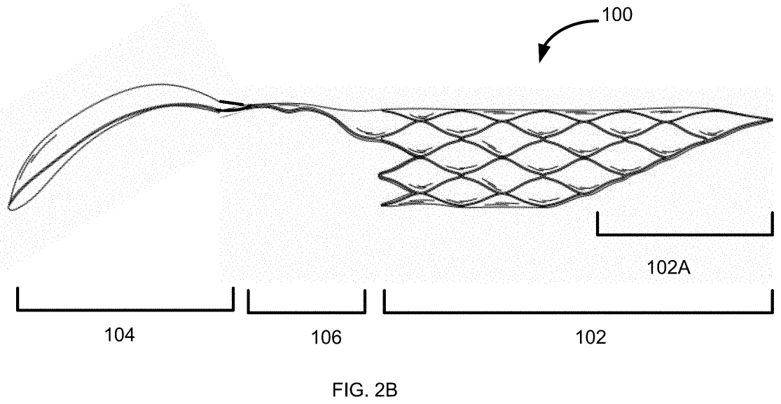

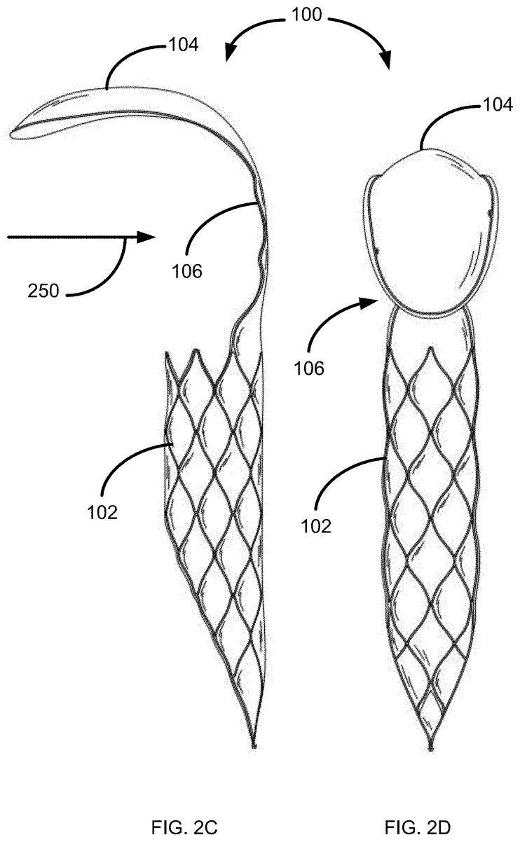

FIG. 1 is a simplified illustration of an exemplary intra-aortic emboli protection filter device 100 deployed in an aorta 10, according to an exemplary embodiment of the invention. FIGS. 2B-2D are additional illustrations of exemplary device 100. In some embodiments, device 100 is configured for placing downstream from the aortic valve, to deflect, filter, and collect emboli particles flowing toward the descending aorta and into branching arteries. As used herein, the terms “upstream” and “downstream” refer to an orientation relative to the direction of flow in a blood vessel.

FIGS. 2C and 2D are side view and front view simplified illustrations of intra-aortic emboli protection filter device 100 in a deployed state as positioned in the aortic arch as shown in the exemplary embodiment illustrated in FIG. 1. FIG. 2D is viewed from a direction indicated in FIG. 2C by arrow 250.Device 100 may include an anchor 102 to be located along descending aorta 13, and a deflector 104 located upstream from anchor 102, within the aortic arch 10. Deflector 104 may be connected to anchor 102 with a connecting section 106. Device 100 is configured for delivery into position at a treatment site in a compressed delivery state, inside a sheath or lumen of a delivery device, such as a catheter. Following release, device 100 is configured for self-expanding within the body vessel or lumen.

In some embodiments, deflector 104 is configure for placing within the aortic arch 10 by being pushed via connecting section 106 against the superior wall of the aortic arch. Deflector 104 comprises a filter screen region having an elongated generally convex shape selected to approximately track the curvature of a selected portion of the aortic arch; cover entrances to arteries 12 branching superiorly from the aorta; guide the tips of instruments introduced into the aortic arch; and deflect embolic particles released during a procedure downstream.

Anchor 102 extends downstream from deflector 104 within the descending aorta, and is connected to deflector 104 with a connecting section 106. Anchor 102 is configured for expanding within the body lumen following release from the delivery device, so as to radially push against walls of the descending aorta, thus anchoring the device in a desired location. In some embodiments, anchor 102 further includes two or more internal filter pockets having each an opening directed upstream, configured for capturing and collecting emboli particles from the blood stream. Anchor 102 may be configured for preventing emboli deflected by deflector 104 from flowing downstream into any arteries 14 which may branch off a continuation of the descending aorta 13.

In some embodiments, device 100 or portions of it form a protective layer along the inner perimeter of the aorta (when in the expanded configuration) for protecting the aorta from damage by an interventional instrument, such as a catheter, wire, valve delivery system, and/or the like, when entering and/or moving along the aorta. For example, the material of the device or those portions thereof may have a low coefficient of friction, or be coated with a low coefficient of friction material, to facilitate the surgical instrument in moving freely along the aortic arch.

In some embodiments, device 100 may be structured as a flexible frame formed of a suitable filament material, such as Nitinol, another shape-memory metal alloy, a metal spring alloy, stainless steel, titanium or titanium alloy, a super-elastic material, and/or a bio-compatible polymer.

In some embodiments, the frame structure of device 100 comprises oval, diamond, or similar zig zag-type cell-like construction, configured for (i) allowing directional collapse of the device into a reduced diameter around its longitudinal axis, so as to fit into a sheath or catheter of the delivery and retrieval device, (ii) retaining the original shape of the device without taking a permanent set, creep, and/or deformation during containment within the delivery device, and (ii) following release from the delivery device, self-expanding and substantially conforming to the overall shape and local topology of selected areas of the blood vessel.

In some embodiments, device 100 may be integrally formed, or otherwise comprises several parts. Device 100 may further be manufactured using laser cutting, or through forming, braiding, pressing, heat-treating, shaping, and the like.

In some embodiments, at least portions of device 100, including anchor 102, deflector 104, and connecting section 106, comprise and/or are lined with filter material configured for filtering and capturing emboli. As used herein, the term “filter” refers to any porous membrane, woven fabric or mesh, or another suitable filtering structure and/or material, that is able to allow certain components of a mixture to pass through, while retaining or deflecting other components. For example, the filter material of the present device may include a blood-permeable material comprising pores, holes, or apertures of a specified size and/or shape, to allow blood to pass through largely unimpeded, while preventing emboli from passing through the blood permeable material. In some embodiments, materials used for the filter include one or more of: polymer such as Polyurethane (PU), Nylon, Nitex, Peektex, Polyester (PET) Polypropylene (PP); a woven, knit, or knotted material with holes; and other materials with holes which can serve as a filter.

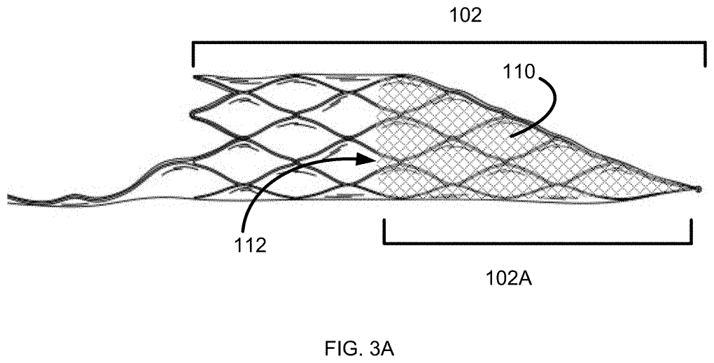

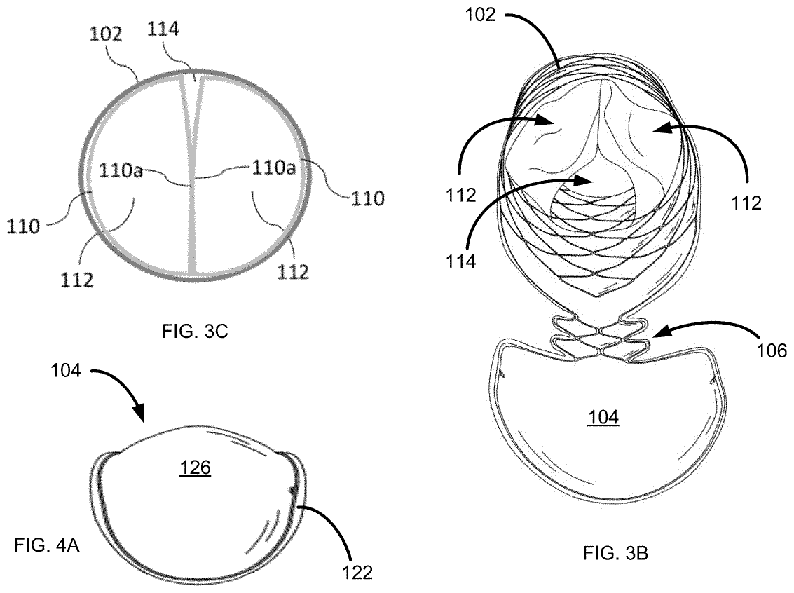

FIGS. 3A-3C show a side view (3A) and downstream views (3B-3C) of anchor 102. In some embodiments, anchor 102 comprises a cylinder-like frame structure that is obliquely-cut at a downstream end 102a. In some embodiments, the frame structure of anchor 102 is configured for expanding radially against walls of the descending aorta (not shown in FIGS. 3A-3C), thus (i) anchoring device 100 in a desired location, and (ii) potentially producing an effective seal between the outer peripheral surface of anchor 102 and the walls of the descending aorta.

In some embodiments, the removal of a section from the cylinder-like structure to form obliquely-cut area 102a provides a region of expansion for debris-collecting filter pockets 110 (further describe below), as they collect emboli particles from the blood stream. This may also provide an advantage when collecting device 100 back into a catheter at the conclusion of the procedure, when the pockets have trapped particles in them.

In some embodiments, anchor 102 is lined with a filter material along at least parts of its inner surfaces. In some embodiments, anchor 102 further comprises at least two elongated filter pockets 110 arranged side-by-side and attached internally to anchor 102, and configured for capturing and retaining the emboli particles flowing downstream.

In some embodiments, filter pockets 110 have each a generally semicircular opening 112 directed upstream, wherein both openings 112 are configured for jointly covering substantially the entire cross-sectional area of anchor 102, and thus, of the body lumen section in which anchor 102 in positioned. In some embodiments, filter pockets 110 are arranged to provide for a gap 114 which enables a surgical tool to pass through anchor 102, between filter pockets 110, wherein regions of pockets 110 continue to form a seal around the periphery of the surgical instrument.

Openings 112 are generally located in an upstream area of anchor 102 where the frame structure has a substantially circular cross-section. In some embodiments, flap receptacle portions of pockets 110 having closed ends extend downstream from openings 112 into obliquely-cut area 102a, and are configured for creating expandable receptacles within anchor 102. In some embodiments, pockets 110 generally taper in the downstream direction. In some embodiments, pockets 110 extend downstream to a tip of obliquely-cut area 102a.

In some embodiments, parts of outer surfaces of pockets 110, including parts of the perimeters of openings 112, are attached to the frame structure of anchor 102. Attaching parts of the perimeters of openings 112 to the structure in this manner ensures that openings 112 remain normally at least partially non-collapsed. This, in turn, ensures that, when placed within the descending aorta, blood flow will be able to enter the at least partially-open openings 112 and cause pockets 110 to further expand into a fully-opened state. This, in turn, ensures that openings 112 cover substantially the entire cross-sectional area of anchor 102, and provide for filtration of the entire blood stream flowing through the blood vessel.

In some embodiments, filter pockets 110 are attached to the frame structure of anchor 102 by being sewn onto it at multiple points. In other examples, filter pockets 110 may be fused and/or glued to the frame structure of anchor 102. In some embodiments, adjoining inner walls of the flap receptacle (reference numbers 110a in FIG. 3C) are at least partially attached to each other by gluing or fusing, e.g., using a thermoplastic material which seeps through the pores of the filter material and fuses the adjoining areas together.

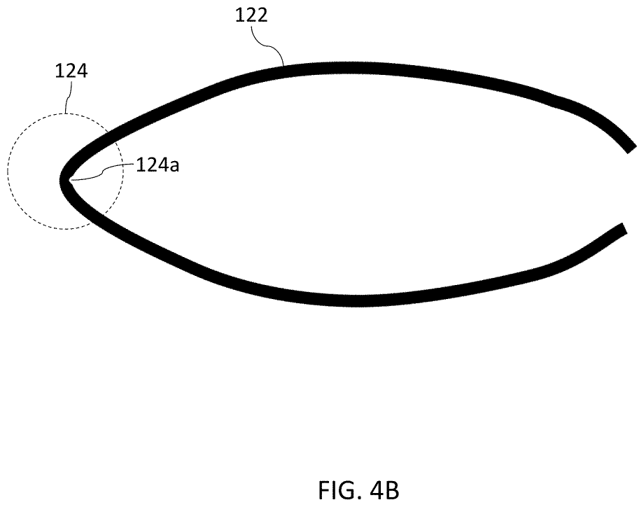

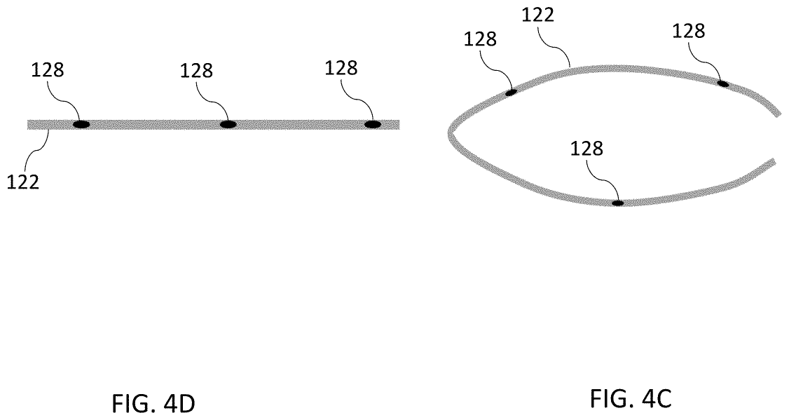

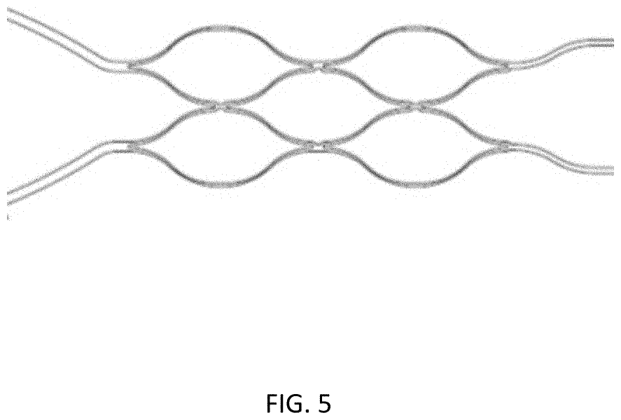

FIG. 4B is a simplified illustration of a frame 122 of an exemplary deflector 104. In some embodiments, frame 122 is an elongated substantially oval frame. Frame 122 may be integrally formed with the structure of device 100, or otherwise separately formed of a suitable filament or strand material, such as Nitinol, another shape-memory metal allow, a metal spring alloy, a super-elastic material, and/or a bio-compatible polymer. In some embodiments, frame 122 comprises one or more notches, such as notch 124a, at one or more corresponding internal corners 124 of frame 122, which notches 124a are configured for facilitating collapsing of frame 112 along its longitudinal axis into a delivery or retrieval state.

FIG. 4A shows an exemplary deflector 104. In some embodiments, deflector 104 comprises a deflector screen 126 attached along a periphery of frame 122. Deflector screen 126 may be formed as a three-dimensional structure resembling an elongated trough with a generally ridge-like cross-section, which elongated trough further curves concavely along a longitudinal axis of frame 122. Thus, deflector screen 126 is configured for tracking approximately the longitudinal and cross-sectional concavities of a desired portion of the superior wall of the aortic arch. When in position, the elongated trough of deflector screen 126 is further configured for providing a track along which instruments passing into the aortic arch from the descending aorta can slide. In some embodiments, wall portions of the elongated trough of deflector screen 126 are configured for urging an instrument passing along the track towards the longitudinal center of the track, thus minimizing the chance that the instrument will slip laterally over the banks of the track. In some embodiments, the contact surface of the trough may further include a low-friction surface to further facilitate the sliding of the instrument.

In some embodiments, deflector screen 126 is integrally formed of a single sheet of filter material, and is, e.g., heat-shaped or otherwise molded into a desired shape. In other embodiments, deflector screen 126 is formed of two or more individual pieces of filter material which are sewn, glued, and/or fused together along their seams to obtain the desired shape.

In some embodiments, deflector screen 126 is attached to frame 122 along its perimeter, e.g., by sewing, gluing, and/or fusing it to frame 122. For example, in some embodiments, portions of the filter material may be attached to a filament of the structure by wrapping the filter material around the filament and fusing, gluing, or sewing its edges together. In some embodiments, fusing of layers of filter material may be achieved by using a thermoplastic or another adhesive material which seeps through pores of the filter material and fuses the layers together.

FIGS. 4C-4D show simplified top and side views of an exemplary frame 122 comprising radio opaque markers 128. In some embodiments, frame 122 may comprise three or more radio-opaque markers 128 configured for assisting in locating the positioning of deflector 104 using imaging systems. As shown in FIG. 4D, when deflector 104 is properly oriented within the aortic arch, radio opaque markers 128 are aligned along the same line.

With reference back to FIGS. 1B-1C, in some embodiments, connecting section 106 comprises an elongated connection section having a frame structure similar to that of other parts of device 100. In some embodiments, connecting section 106 is designed and shaped to connect anchor 102 and deflector 104, so as to urge deflector 104 lie against the walls of the aortic arch. Another potential benefit of connecting section 106 is to distance the anchor from the more tortuous part of the descending aorta to a relatively less tortuous part of the descending aorta, so that the device potentially causes less interference with a procedure device, especially with a valve delivery system. Another potential benefit of the connecting section 106 is to protect the aortic wall from potentially harmful interaction by the valve delivery system.

In some embodiments, connecting section 106 includes a frame lined with filter material. In some embodiments, the filter has identical or similar characteristics to the filter of the pockets 110 and deflector screen 126. In some embodiments connecting section 106 is produced in different sizes, to fit different sizes and shapes of descending aorta. In some embodiments a length of connecting section 106 is in a range from 2 cm to 20 cm, and a width of connecting section 106 is in a range from 5 mm to 6 cm. In some embodiments, the frame structure of connecting section 106 is configured for facilitating directional collapse of connecting section 106 along it length. In some embodiments, connecting section 106 has an arched or semi-arched cross-sectional profile. In some embodiments, the width, the number of longitudinal filaments comprising the mesh structure, and/or the cross-sectional profile of connecting section 106 combine to increase the structural rigidity of connecting section 106. Accordingly, connecting section 106 is able advance deflector 104 into the aortic arch, while maintaining a proper orientation of deflector 104 and preventing, e.g., its rotation about its longitudinal axis. In some embodiments connecting section 106 is optionally pre-shaped to accommodate to the curvature of the aorta.

In some embodiments, the filter material of the present device comprises one layer of material. In some embodiments, the filter material of the present device is made of a double layer of material. In some embodiments, any portion of the present device e.g., the deflector filter material may be shaped e.g., into a spoon-like shape. In some embodiments, one or more sheets of filter material are assembled into a mold that is lightly stretched to receive the mold shape. In some embodiments, one or more filter mesh sheets are pressed and heated for a predetermined period of time to fix the spoon-like shape of the filter mesh.

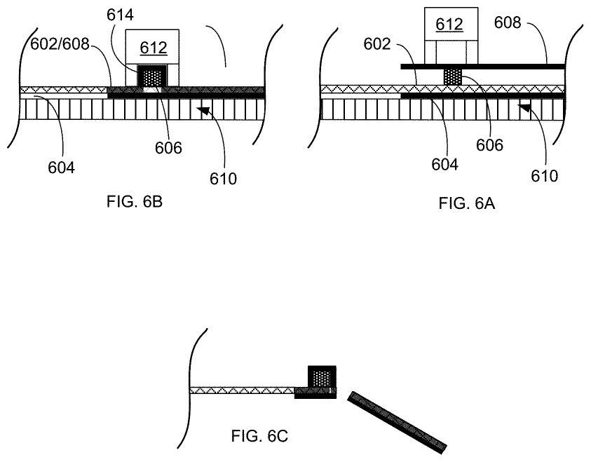

In some embodiments, the filter mesh is fixed to the frame by welding. In some embodiments, and as shown in the exemplary embodiment depicted in FIGS. 6A, 6B and 6C, which are cross-section view simplified illustrations of a welding configuration of the filter mesh to the intra-aortic emboli protection filter device 100 frame, the frame and filter material are assembled as follows: A sheet of filter mesh 602 is layered (FIG. 6A) over a first sheet of a polymer material 604 (e.g., Polyurethane, PU). the frame 606 is placed on top of the sheet of the filter mesh 602 and covered by a second sheet of PU 608. The layered construction is placed in a dedicated tool (FIG. 6B) e.g., between an anvil 610 and a welding knife 612, and pressure and heat are applied over PU areas along both sides of the frame such as to form a sleeve of polymer material 614 in which the frame 606 is accommodated. Under the heat and pressure, first PU layer 604 bonds into mesh 606 and second PU layer 608 to form a uniform bond forming a sleeve 616 in which frame 606 is accommodated.

The welding assembly then cooled and removed and excessive filter material, e.g., around the periphery of the deflector frame, is cut and removed (FIG. 6C).

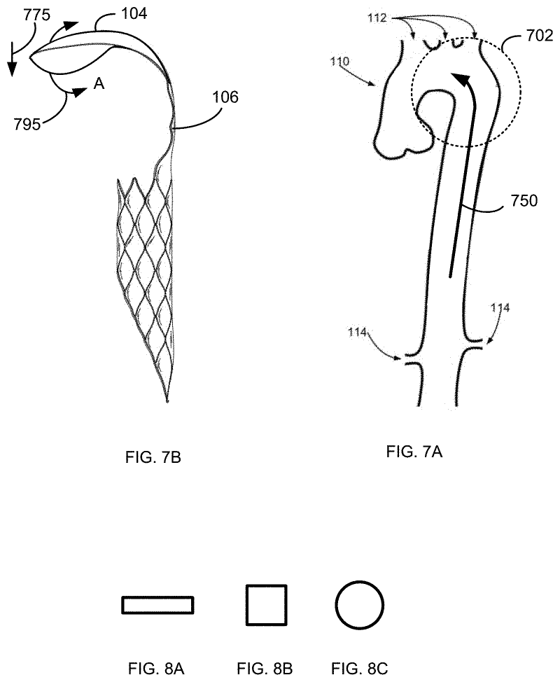

Reference is now made to FIG. 7A and 7B, which are perspective view and cross-section view simplified illustrations of an intra-aortic emboli protection filter device (7A) and a cross-section of an aorta (7B) and FIGS. 8A, 8B and 8C, which are cross-sections of an intra-aortic emboli protection filter device frame wire in accordance with some embodiments of the current invention.

In some embodiments, and as shown in FIG. 1, intra-aortic emboli protection filter device 100 is deployed in an aorta by advancing the device 100 deployment catheter in a direction indicated in FIG. 7A by an arrow designated reference number 650. When passing device 100 into the aortic arch, encircled in FIG. 6A by a circle 702, the wall of the aortic arch urges deflector 104 downwards as indicated in FIG. 7B by an arrow 775. It is not uncommon at this stage for deflector section 104 of device 100 to twist about a longitudinal axis of device 100 in a clockwise or anticlockwise direction as indicated in FIG. 7B by double-headed arrow 795. In the exemplary embodiment shown in FIG. 7B, a portion of deflector 104 is twisted in a direction indicated by reference letter (A). This phenomenon places torque stress on connecting section 106 as well as possibly only partially blocking openings 112.

This phenomenon is commonly seen when a device 100 frame wire comprises a flattened cross-section for example, a cross-section shown in FIG. 8A. Square (FIG. 8B) or round (FIG. 8C) cross-sections of device 100 frame wire eliminate this phenomenon.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An embolic protection filter device comprising:
a deflector comprising a filter screen;
an anchor comprising a cylinder-like frame having at least one filter pocket attached internally thereto, said filter pocket having an opening directed upstream; and
a connecting section configured for connecting said deflector and said anchor.

2. The device of claim 1, wherein said device comprises an integrally-formed frame structure, said frame structure being configured for collapsing, in a delivery state, into a reduced diameter configuration around a longitudinal axis thereof, and wherein said frame structure comprises a plurality of filaments arranged in a cell-like construction.

3. (canceled)

4. The device according to claim 2, wherein said frame structure is made of one or more materials selected from the group consisting of: Nitinol, shape-memory metal alloy, metal spring alloy, stainless steel, titanium, titanium alloy, super-elastic material, and bio-compatible polymer.

5. The device according to claim 1, wherein said deflector is configured for positioning within an aortic arch and for conforming to a superior wall of the aortic arch.

6. The device according to claim 1, wherein said deflector is formed as an elongated trough which curves along a longitudinal axis thereof, and wherein a longitudinal center of said elongated trough is configured for providing a track for guiding surgical instruments traversing the aortic arch.

7. (canceled)

8. The device according to claim 1, wherein said deflector further comprises a support frame, said support frame having an elongated oval-like shape, and wherein said support frame further comprises one or more notches at corresponding one or more internal corners thereof, said notches being configured for facilitating collapsing of said support frame along said longitudinal axis into said delivery state.

9. (canceled)

10. (canceled)

11. The device according to claim 8, wherein said support frame further comprises at least three radio-opaque markers at specified points along said perimeter, and wherein an orientation of said positioning of said deflector within the aortic arch can be determined based, at least in part, on imaging said at least three radio-opaque markers.

12. (canceled)

13. The device according to claim 1, wherein said cylinder-like frame of said anchor comprises an obliquely-cut section at a downstream end thereof.

14. The device according to claim 1, wherein said anchor comprises at least two filter pockets arranged longitudinally side-by-side internally thereof, wherein said at least two filter pockets are configured for trapping emboli flowing downstream through said anchor, and wherein said trapping causes an expansion of said at least two filter pockets.

15. (canceled)

16. The device of claim 14, wherein said cylinder-like frame of said anchor comprises an obliquely-cut section at a downstream end thereof, and wherein said expansion occurs, at least in part, within said obliquely-cut section.

17. The device of claim 14, wherein said openings of said at least two filter pockets are each attached along at least a portion of its perimeter to said cylinder-like frame, and wherein said attaching causes said openings to maintain at least a partially-opened state.

18. The device of claim 17, wherein blood flow through said openings, in said least partially-opened state, further causes said openings to achieve a fully-opened state, and wherein, in said fully-opened state, said openings are configured for jointly covering substantially a cross-sectional area of said cylinder-like frame.

19. (canceled)

20. The device of claim 14, wherein adjoining walls of said at least two filter pockets are at least partially attached to each other, and wherein said attaching of said adjoining walls is further configured to enable a surgical tool to pass through a gap between said adjoining walls, and wherein said gap is dimensioned to effect a seal around a periphery of said surgical tool.

21. (canceled)

22. (canceled)

23. (canceled)

24. (canceled)

25. (canceled)

26. A method for protecting a patient against flow of emboli from an aorta to branching arteries, the method comprising:
providing a device comprising:
a deflector comprising a filter screen,
an anchor comprising a cylinder-like frame having at least one filter pocket attached internally thereto, said filter pocket having an opening directed upstream, and
a connecting section configured for connecting said deflector and said anchor; and
inserting said device via a catheter into an aorta, such that:
said deflector screen is positioned at the aortic arch, and
said anchor is positioned along the descending aorta.

27. (canceled)

28. The method according to claim 26, wherein said positioning of said deflector at the aortic arch further comprises pushing said deflector, via said connecting section, against a superior wall of the aortic arch.

29. The method of claim 28, wherein said deflector further comprises a support frame comprising at least three radio-opaque markers at specified points along a perimeter thereof, the method further comprising the step of determining an orientation of said positioning of said deflector within the aortic arch based, at least in part, on imaging said at least three radio-opaque markers.

30. The method according to claim 26, wherein said anchor comprises at least two filter pockets arranged longitudinally side-by-side internally thereof, and wherein adjoining walls of said at least two filter pockets are at least partially attached to each other, so as to enable a surgical instrument to pass through a gap between said adjoining walls, the method further comprising performing a procedure on a heart of the patient by passing said surgical instrument through said gap.

31. The method of claim 30, wherein said deflector is formed as an elongated trough which curves along a longitudinal axis thereof, the method further comprising using a longitudinal center of said elongated trough as a track for guiding said surgical instruments while traversing the aortic arch.

32. The method according to claim 26, further comprising the step of collecting said device into said catheter, wherein said collecting comprises collapsing said device into a delivery state wherein said device has a reduced diameter configuration around a longitudinal axis thereof.

33. The method according to claim 26, wherein said positioning of said deflector further comprises covering entrances of arteries branching superiorly from the aorta.
